(19) United States
(12) Patent Application Publication
(10) Pub. No.: US 2024/0260859 A1
(43) Pub. Date: Aug. 8, 2024

(54) DETECTION OF A CHEMICAL SPECIES IN THE SWEAT OF A SUBJECT

(71) Applicant: NOPTRACK, Castres (FR)

(72) Inventors: Christian AMATORE, Paris (FR); Gilles FAVRE, Cugnaux (FR); Fethi BEDIOUI, Paris (FR); Yong CHEN, Paris (FR); Sophie GRIVEAU, Massy (FR); Catherine SELLA, Meudon la forêt (FR); Laurent THOUIN, Antony (FR)

(21) Appl. No.: 18/565,808

(22) PCT Filed: May 31, 2022
(86) PCT No.: PCT/EP2022/064783

§ 371 Date: Nov. 30, 2023

(30) Foreign Application Priority Data
Jun. 2, 2021 (FR) FR2105827

(51) Int. Cl. A61B 5/145; A61B 5/00; A61B 5/1477; A61B 10/00

(52) U.S. Cl. CPC A61B 5/14517; A61B 5/1477; A61B 10/0064; A61B 5/6803; A61B 5/6804; A61B 5/681; A61B 5/6898; A61B 2560/0468; A61B 2562/0215; A61B 2562/028; A61B 2562/0295

(57) ABSTRACT

A detection apparatus for placement on an investigation zone of an epidermis of a human or animal subject for detecting at least the nitric oxide dissolved in sweat, said apparatus comprising:
a structure (1) defining a microfluidic circuit for guiding a flow of sweat, the structure comprising an entry orifice (4) allowing passage of sweat from the epidermis, the microfluidic circuit comprising at least one microfluidic channel (9) in communication with the entry orifice, at least one electrochemical sensor (10) comprising at least four electrodes disposed successively in a longitudinal direction of the microfluidic channel, the at least four electrodes comprising a reference electrode, at least two working electrodes and a counter-electrode,
the electrochemical sensor being configured to produce a signal that is representative of a concentration of nitric oxide and further for implementing a depletion and/or for producing a signal that is representative of a flow rate of the flow of sweat.

### TECHNICAL FIELD

The invention relates to methods and apparatuses for detecting and measuring chemical species dissolved in the sweat of a human or animal subject, more particularly nitric oxide.

### TECHNOLOGICAL BACKGROUND

Nitric oxide is a gas which constitutes an intercellular messenger. It is an important cardiovascular messenger of stress by mechanotransduction. It is emitted more particularly for stimulating the vasodilation of the vascular system during muscular exertions. Variations in the flow of the gas produced in the blood and therefore in the liquids in equilibrium with the blood, for example sweat, therefore constitute a particularly relevant indicator of the cardiovascular capacity of a patient to adapt to the muscle power required during exertion tests.

Accordingly, there is a genuine need to develop an apparatus for detecting nitric oxide (NO) that is easy to manufacture, is reliable, and is easy to use.

Document WO2019229380A1 described an apparatus allowing instantaneous measurement of nitric oxide in the sweat at the epidermis of a subject, and clinical applications thereof.

### SUMMARY

Certain aspects of the invention are based on the idea that quantitative measurement of the variations in the concentration of nitric oxide in sweat provides a non-invasive technique for monitoring the cardiovascular capacity during preventive checks or for establishing a diagnosis.

Certain aspects of the invention are based on the observation that in the presence of dioxygen, nitric oxide reacts spontaneously to produce a nitrite ion (NO₂⁻) via a reaction with an overall stoichiometry of 1:1. In other words, whereas variations in the concentration of nitric oxide represent the present state of the responses of the cardiovascular system to a given exertion, variations in the concentration of the nitrite ion constitute a temporal record of these responses.

Certain aspects of the invention are based on the idea of detecting a plurality of chemical compounds in a coupled manner by an integrated electrochemical device.

Certain aspects of the invention are based on the idea that variations in concentrations of nitric oxide and of the nitrite ion may be detected and quantified in a coupled manner by an integrated electrochemical device.

Certain aspects of the invention are based on the observation that nitric oxide is produced by specialized enzymes (NO synthases) from the breakdown of intracellular L-arginine in the presence of dioxygen (O₂) and an electron source. When the availability of L-arginine decreases because of a high level of consumption (for example, following prolonged exertion) or because of chronic deficiency, the NO synthases continue to react with the oxygen, limiting themselves to reducing the dioxygen to superoxide ion (O₂). The latter spontaneously changes very rapidly into hydrogen peroxide (H₂O₂), via a reaction with an overall stoichiometry of 2:1. Certain aspects of the invention are based on the idea that the presence of detectable concentrations of hydrogen peroxide in the blood, and therefore in sweat, provides an indicator representing a level of distress of the cardiovascular network. Moreover, in the presence of metal salts, hydrogen peroxide decomposes to form highly toxic radical species (HO·, HO₂·, etc.) which are capable of bringing about very great damage to the cells of the cardiovascular system, including those of the heart. Certain aspects of the invention are based on the idea that detecting production of hydrogen peroxide in parallel with detecting production of nitric oxide and/or nitrite ion is relevant for evaluating the cardiovascular capacities of a patient.

Certain aspects of the invention are based on the idea of measuring the quantity at least of the nitric oxide by an electrochemical device and of the nitrite ion by a colorimetric technique.

Certain aspects of the invention are based on the observation that the physiological system of a subject is dynamic, since the volume flow rate of sweat may vary for adjusting the capacity for removal of the heat energy produced as a function of the muscle power delivered. The exchange flows of each chemical species at the interfaces between the blood and sweat may vary as a function of an exertion provided by the subject. Certain aspects of the invention are based on the idea of quantitatively and dynamically detecting the production of one or more of the chemical species selected from nitric oxide, nitrite ion, hydrogen peroxide and optionally peroxynitrite, for example during an exertion test or medical monitoring of the subject.

To this end, the invention provides a detection apparatus for placement on an investigation zone of an epidermis of a human or animal subject for detecting at least the nitric oxide dissolved in sweat, said detection apparatus comprising:
a structure defining a microfluidic circuit for guiding a flow of sweat, the structure comprising an entry orifice allowing passage of sweat from the epidermis, the microfluidic circuit comprising at least one microfluidic channel in communication with the entry orifice, at least one electrochemical sensor comprising at least four electrodes disposed successively in a longitudinal direction of the microfluidic channel, the at least four electrodes comprising a reference electrode, at least two working electrodes and a counter-electrode,
the electrochemical sensor being configured to produce at least one signal that is representative of a concentration of the nitric oxide dissolved in the flow of sweat and being further configured to perform at least one additional operation from among the following:
depleting a chemical species in the flow of sweat, said chemical species having an oxidation potential lower than the oxidation potential of nitric oxide, and
producing a signal that is representative of a flow rate of the flow of sweat.

By virtue of these characteristics, it is possible to reliably measure the concentration of nitric oxide (NO) dissolved in sweat.

An epidermis refers to the surface layer of the skin in humans and animals.

The detection apparatus is non-invasive and does not need to be applied to a wound.

According to embodiments, an apparatus of this kind may comprise one or more of the characteristics below.

According to one embodiment, the structure is a multilayer structure comprising a lower layer and at least one layer atop the lower layer, the microfluidic circuit extending parallel to the lower layer, and the lower layer comprising said entry orifice.

According to one embodiment, the or each or at least one said electrochemical sensor is configured to produce a signal that is representative of the flow rate of the flow of sweat in the microfluidic channel, and hence of a volume flow rate of sweat in the microfluidic channel.

According to one embodiment, the multilayer structure comprises an upper layer and at least one middle layer situated between the lower layer and the upper layer, the microfluidic circuit being formed in the thickness of the at least one middle layer. The layers may be secured to one another by any appropriate technique, for example, by adhesives, by welding, by mechanical clamping, etc.

By virtue of these characteristics, the manufacture, the assembly and therefore the industrialization of the detection apparatus are facilitated.

According to one embodiment, the at least one middle layer comprises a first middle layer and a second, sealing middle layer situated between the first middle layer and the upper layer, the second, sealing middle layer comprising an opening at the electrodes.

By virtue of these characteristics, the detection apparatus is adapted to any curvatures when it is applied to the epidermis. Furthermore, the one or more middle layers also make it possible to create a thickness that allows the thickness of the at least four electrodes to be compensated. The sealing of the detection apparatus is therefore ensured.

According to one embodiment, the at least one middle layer comprises an outlet orifice enabling discharge of the flow of sweat which has undergone the one or more electrochemical measurements.

According to one embodiment, the multilayer structure comprises an upper layer and an outlet orifice traversing the upper layer, wherein the at least one microfluidic channel is in communication with the outlet orifice.

According to one embodiment, the lower layer is coated with an adhesive made of flexible biocompatible material. According to one embodiment, the lower layer is adhesive on a first face, intended for positioning on the skin, and a second face, intended to receive the superposed layer or layers.

By virtue of these characteristics, the detection apparatus measures a concentration of at least the concentration of nitric oxide without disturbing the circulation of sweat in the investigation zone of the subject's epidermis. This means that the detection apparatus operates without affecting the passage of sweat through the epidermis in the investigation zone of the subject's epidermis.

According to one embodiment, the multilayer structure is made from one or more of the following materials:
inorganic materials such as silica, glass and light-sensitive glass;
   elastomeric materials such as polydimethylsiloxane (PDMS), modified polydimethylsiloxane (PDMS) modified with (poly(acrylic acid), poly(ethylene oxide), TiO2, aluminum film, polycations, polyanion, mPEG, sol-gel, amine-thiol-carboxyl alkoxysilanes, surfactants), poly(methyl methacrylate) (PMMA) and polycarbonate (PC);
thermosetting materials such as the light-sensitive resin SU-8;
thermoplastic materials such as polystyrene (PS), polyethylene terephthalate (PET), polyvinyl chloride (PVC), perfluoroalkoxy (Teflon-PFA®), fluoro-ethylene-propylene (Teflon-FEP®);
hydrogel materials such as Matrigel®, collagen, chitosan, alginate, agarose, PEG and polyacrylamide;
paper materials such as cellulose.

According to one embodiment, the multilayer structure comprises at least one layer made of polymer materials. For example, to trace the microfluidic circuit in a polymer material, membrane cutting or molding techniques may be used.

According to one embodiment, the multilayer structure is made of polymer material.

According to one embodiment, the multilayer structure comprises at least one layer made of fibrous materials, for example paper or nonwoven. For example, to trace the microfluidic circuit in a fibrous material, a coating of hydrophobic ink or of hydrophobic resin delimiting the contours of the microfluidic circuit may be used.

By virtue of these characteristics, the multilayer structure is lightweight, flexible and pliant. In particular, this allows the detection apparatus to be able to be used on numerous parts of the body such as the back, the arm, the shoulder, the leg or the nape of the neck, without risk of damage to the apparatus or of reduced detection reliability.

According to one embodiment, the multilayer structure comprises at least one layer made of glass.

With preference, the multilayer structure is made from: polyethylene terephthalate (PET), polyethylene naphthalate (PEN), acrylic adhesive, polybutyrate adipate terephthalate (PBAT), polyurethane layer and polyacrylate layer (MPU).

According to one embodiment, wherein the at least four electrodes are disposed on an inner face of the upper layer closing the microfluidic channel at the top and/or on an upper face of the lower layer closing the microfluidic channel at the bottom.

By virtue of these characteristics, the electrodes of the detecting apparatus are disposed reliably. Furthermore, manufacture of the multilayer structure comprising these electrodes is facilitated, in that it is possible to manufacture the electrodes on a planar layer when the microfluidic circuit is formed in a middle layer.

According to one embodiment, the electrodes consist of applied metals.

According to one embodiment, the applied metals are selected from the group consisting of silver (Ag), gold (Au), platinum (Pt), and platinum black. Graphite or carbon may also be used. According to one embodiment, the electrochemical sensor comprises a silver/silver chloride (Ag/AgCl) or other reference electrode.

According to one embodiment, the electrochemical sensor is configured to deplete a chemical species having an oxidation potential lower than the oxidation potential of nitric oxide. For this purpose, in one embodiment, in the direction of the flow, the at least four electrodes comprise in succession the first working electrode, in the form of a depletion electrode, the second working electrode, for measuring the concentration of nitric oxide, and the counter-electrode, the reference electrode being placed at a position immediately upstream of the first working electrode or immediately downstream of the second working electrode.

By virtue of these characteristics, it is possible to increase the precision of the electrochemical sensor, by greatly reducing spurious signals that may be due to chemical compounds which oxidize at electrical potentials lower than the electrical potential for oxidation of nitric oxide. Accordingly, the set of electrodes acts synergistically to obtain an accurate result.

According to one embodiment, the depletion electrode is wider than the second working electrode required for measuring the concentration of nitric oxide, for example at least four times wider.

By virtue of the large surface area of the depletion electrode, the electrolysis of the flow of sweat passing over the depletion electrode may be substantially complete. Moreover, the phenomenon of electrode passivation is reduced.

According to one embodiment, one or more of the electrodes, for example the depletion electrode, is covered with platinum (Pt) black.

By virtue of these characteristics, the electrode enables catalysis of electrochemical reactions via the imperfect and therefore reactive nature of the Pt black dendrites and prevents deactivation of the bare metal surfaces.

According to one embodiment, the electrochemical sensor is configured to polarize the depletion electrode to an electrical potential enabling oxidation of at least one chemical species selected from: hydrogen peroxide (H2O2), peroxynitrite (ONOO⁻) and optionally other chemical species which oxidize at these potentials.

According to one embodiment, the counter-electrode of the electrochemical sensor has a width at least equal to the sum of the width of the set of electrodes disposed upstream of the counter-electrode.

According to one embodiment, the electrochemical sensor is configured to measure a flow rate of the flow of sweat in the microfluidic channel. For this purpose, according to one embodiment, in the direction of the flow, the at least four electrodes comprise in succession the first working electrode for measuring the concentration of nitric oxide, the second working electrode for measuring the concentration of nitric oxide, and the counter-electrode, the reference electrode being placed at a position immediately upstream of the first working electrode or immediately downstream of the second working electrode.

By virtue of these characteristics, it is possible to measure the flow rate of the flow of sweat between the first working electrode and the second working electrode.

According to one embodiment, the electrochemical sensor is configured to produce the signal that is representative of the flow rate by measuring a delay between a variation in current in the first working electrode and a variation in current in the second working electrode.

According to one embodiment, the distance separating the upstream working electrode and the downstream working electrode is preferably less than the distance covered by the flow in one minute. By virtue of these characteristics, it is possible to obtain a precise measurement of flow rate without possible disturbance due to a physiological change in the subject.

According to one embodiment, the electrochemical sensor is configured to produce a signal that is representative of instantaneous production of nitric oxide in the investigation zone on the basis of the signal that is representative of the concentration of nitric oxide and of the signal that is representative of the flow rate of the flow of sweat.

“Instantaneous production” denotes a measurement taken over a very short time relative to the characteristic time of the variation in physiological response of the subject. This characteristic time is typically of the order of one to several minutes for a human subject.

According to one embodiment, the electrochemical sensor is configured to produce the signal that is representative of the concentration of nitric oxide by an electrical, especially amperometric, measurement between at least one of said working electrodes and the counter-electrode.

According to one embodiment, the electrochemical sensor is configured to polarize at least one of said working electrodes to an electrical potential for oxidizing nitric oxide.

According to one embodiment, the electrochemical sensor is configured to measure the concentrations of one or more other chemical species in the flow of sweat in addition to measuring the concentration of nitric oxide. For this purpose, the electrochemical sensor is configured to produce the signal that is representative of the concentration of the one or more chemical species of interest by an amperometric measurement.

According to one embodiment, the electrochemical sensor is further configured to produce a signal that is representative of a concentration in the flow of sweat of at least one of the following chemical compounds: nitrite ion, hydrogen peroxide and peroxynitrite, dissolved in sweat.

For this purpose, according to one embodiment, the electrochemical sensor comprises a third working electrode between the first or second working electrode and the counter-electrode, for measuring the chemical compound.

According to one embodiment, the electrochemical sensor is configured to measure the concentration in the flow of sweat of multiple chemical compounds in series in increasing order of the electrical potentials for oxidation of the chemical compounds.

According to one embodiment, the detection apparatus further comprises a colorimetric detection device coupled to the microfluidic circuit.

According to one embodiment, a colorimetric detection device connected to the channel downstream of the electrochemical sensor, the colorimetric detection device comprising a hydrophilic porous body impregnated with a chemical reagent capable of reacting with one of the following chemical compounds: nitrite ion, hydrogen peroxide, peroxynitrite, sulfur dioxide, hydrogen sulfide, nitric oxide, carbon monoxide and hypochlorous acid, dissolved in sweat, so as to provide a colored indicator indicating a quantity of said chemical compound in the flow of sweat.

According to one embodiment, the hydrophilic porous body is selected from: microporous membrane, paper, fabric, cellulose wadding, nonwoven, etc.

According to one embodiment, the chemical reagent comprises a Griess reagent capable of reacting with the nitrite ion dissolved in the flow of sweat.

According to one embodiment, the colorimetric detection device is disposed in the outlet orifice.

According to one embodiment, the microfluidic circuit comprises a plurality of microfluidic channels connected in derivation from one another to the entry orifice.

According to embodiments described below, the detection apparatus is implemented so as to be able to simultaneously or sequentially detect a plurality of chemical species, for example two, three, four or five species, including nitric oxide, with one or more electrochemical sensors. According to one embodiment, the detection apparatus is implemented so as to be able to detect the concentration of the chemical species selected from nitric oxide NO, nitrite ion NO₂⁻ and hydrogen peroxide H₂O₂.

According to this embodiment, the detection apparatus comprises three parallel microfluidic channels fed in parallel by the same entry orifice.

According to one embodiment, the lower layer comprises a plurality of entry orifices and the microfluidic circuit comprises a plurality of independent microfluidic channels connected respectively to each entry orifice.

According to one embodiment, the detection apparatus comprises a fibrous body for guiding sweat from the investigation zone to the entry orifice or to the entry orifices by capillary action. A fibrous body of this kind may be a woven or non-woven material.

According to one embodiment, the plurality of microfluidic channels comprises an additional microfluidic channel comprising an electrochemical sensor, the electrochemical sensor comprising at least three electrodes disposed successively in a longitudinal direction of the additional microfluidic channel, the at least three electrodes comprising a reference electrode, a counter-electrode and at least one working electrode, the additional electrochemical sensor being configured to polarize the electrodes to an electrical potential for oxidation of a chemical compound selected from nitrite ion, hydrogen peroxide and peroxynitrite and being configured to produce at least one signal that is representative of a concentration of said chemical compound dissolved in the flow of sweat.

According to one embodiment, the electrochemical sensor is configured to polarize at least one said working electrode during a determined time with a periodic recurrence.

By virtue of these characteristics, it is possible to obtain a measurement of the concentration of nitric oxide dissolved in the flow of sweat, while reducing the phenomenon of electrode passivation.

According to one embodiment, the electrodes are polarized during a time of between 1 second and 500 seconds with a recurrence having a periodicity of between 1 minute and 60 minutes.

By virtue of these characteristics, it is possible to measure the concentration of a plurality of chemical compounds.

According to one embodiment, a layer of polyeugenol (4-allyl-2-methoxyphenol), of another polyphenol or of a similar polymer is applied to at least one working electrode of the electrochemical sensor. The layer is preferably applied electrochemically.

Other embodiments of the electrochemical sensors will be described below.

According to one embodiment allowing sequential detection, the or each or at least one said electrochemical sensor is configured to sequentially detect multiple chemical species during a plurality of measurement steps, the electrochemical sensor being configured to polarize the electrodes to an electrical potential for oxidation of hydrogen peroxide H₂O₂ during a first step and to polarize the electrodes to an electrical potential for oxidation of nitric oxide NO during a second step, and the electrochemical sensor is configured to produce a signal that is representative of a concentration of nitric oxide NO on the basis of a first amperometric measurement signal obtained in the first step and of a second amperometric measurement signal obtained in the second step.

Advantageously in this case, the or each or at least one said electrochemical sensor is configured to polarize the electrodes to an electrical potential for oxidation of the nitrite ion NO₂⁻ during a third step, and the electrochemical sensor is configured to produce a signal that is representative of a concentration of the nitrite ion NO₂⁻ on the basis of said first and second amperometric measurement signals and of a third amperometric measurement signal obtained in the third step.

According to one embodiment, the detection apparatus is implemented so as to be able to sequentially detect three of the aforesaid chemical species with a single electrochemical sensor during a plurality of steps of temporal measurement sequences. According to this embodiment, in a given sequence, the electrochemical sensor is configured to polarize a platinized (platinum black) platinum electrode sequentially to the electrochemical potential for oxidation of hydrogen peroxide H₂O₂ during a first temporal step of a few seconds (5 s, for example), then to that for the oxidation of nitric oxide NO during a second temporal step with the same duration, and optionally to that of the nitrite ion NO₂⁻ in a third temporal step with the same duration. The electrochemical sensor is configured to produce a signal that is representative of a concentration of nitric oxide NO on the basis of a first amperometric measurement signal obtained in the first step and of a second amperometric measurement signal obtained in the second step. This sequence is repeated as many times as necessary throughout the duration of the exertion test. The resolution of a system of three equations (the currents measured sequentially on the electrode polarized to each potential in a sequence) having two or three unknowns (the concentrations of H₂O₂, NO and NO₂⁻) yields the values of each of the three concentrations at the moment each sequence is implemented on the basis of the three measurements.

According to another embodiment allowing simultaneous detection, the detection apparatus comprises:
a first microfluidic channel coupled to the entry orifice for guiding a first flow of sweat from the investigation zone and a first electrochemical sensor comprising electrodes disposed in the first microfluidic channel, the first electrochemical sensor being configured to polarize the electrodes to an electrical potential for oxidation of hydrogen peroxide H₂O₂, and
a second microfluidic channel coupled to the entry orifice for guiding a second flow of sweat from the investigation zone and a second electrochemical sensor comprising electrodes disposed in the second fluidic circuit, the second electrochemical sensor being configured to polarize the electrodes to an electrical potential for oxidation of nitric oxide NO,
and the electrochemical sensor is configured to produce a signal that is representative of a concentration of nitric oxide NO on the basis of a first amperometric measurement signal produced by the first electrochemical sensor and of a second amperometric measurement signal produced by the second electrochemical sensor.

Advantageously in this case, the detection apparatus further comprises:
a third microfluidic channel coupled to the entry orifice for guiding a third flow of sweat from the investigation zone and a third electrochemical sensor comprising electrodes disposed in the third fluidic circuit, the third electrochemical sensor being configured to polarize the electrodes to an electrical potential for oxidation of the nitrite ion NO₂⁻, and the electrochemical sensor is configured to produce a signal that is representative of a concentration of the nitrite ion NO₂⁻ on the basis of said first and second amperometric measurement signals and of a third amperometric measurement signal produced by the third electrochemical sensor.

According to one embodiment, the detection apparatus further comprises:
another microfluidic channel, for example a fourth microfluidic channel, coupled to the entry orifice for guiding another flow of sweat from the investigation zone, for example fourth flow of sweat, and another electrochemical sensor, for example fourth electrochemical sensor, comprising electrodes disposed in the fourth microfluidic channel.

According to this embodiment, said other or fourth electrochemical sensor is configured to polarize the electrodes to an electrical potential for oxidation of nitric oxide and the fourth microfluidic channel comprises an applied layer of polyeugenol on the working electrode of the fourth electrochemical sensor so as to remove hydrogen peroxide in particular.

According to one embodiment, the apparatus further comprises a layer of adhesive material covering a lower face of the lower layer of the multilayer structure without covering the entry orifice, so as to form an impervious barrier around the investigation zone by contact with the epidermis of said subject.

By virtue of these characteristics, the gases, liquids and microorganisms such as bacteria or viruses that are situated outside of the investigation zone are unable to enter the investigation zone. The imperviousness of the contact between the casing and the epidermis ensures that the chemical species detected comes from the biological liquid produced by the investigation zone, and not from a flow from the outside.

According to one embodiment, the plurality of microfluidic channels comprises an additional microfluidic channel comprising a colorimetric detection device, the colorimetric detection device comprising a hydrophilic porous body impregnated with a chemical reagent capable of reacting with one of the following chemical compounds: nitrite ion, hydrogen peroxide, peroxynitrite, sulfur dioxide, hydrogen sulfide, nitric oxide, carbon monoxide and hypochlorous acid, so as to provide a colored indicator indicating a concentration or a quantity of the chemical compound dissolved in the flow of sweat.

By virtue of these characteristics, the concentration may be monitored over time, making it easier to read the use. Moreover, this allows the results obtained via the electrochemical sensor to be consolidated.

According to one embodiment, the additional channel comprises a chrono-sampling system connected to the entry orifice, the chrono-sampling system including a plurality of chambers configured to fill sequentially with sweat, and in which a plurality of colorimetric detection devices are disposed in said chambers, each colorimetric detection device comprising a chemical reagent capable of reacting with a chemical compound, such that the colorimetric detection devices disposed in said chambers provide a colored indicator indicating a cumulative quantity of said chemical compound in the flow of sweat.

One appropriate chrono-sampling system is described in particular in the Choi et al. document “Thin, Soft, Skin-Mounted Microfluidic Networks with Capillary Bursting Valves for Chrono-Sampling of Sweat, Adv. Healthcare Mater. 2017”.

The structures of the detection apparatus described above may be realised via various methods, for example in the form of multilayer structures. They may also be obtained by additive manufacturing, 3D printing, laminating, or addition of material in successive layers.

According to one embodiment, the detection apparatus further comprises an optical sensor configured to produce a measurement signal that is representative of the intensity of a color of the chemical reagent in the visible or ultraviolet spectrum.

According to one embodiment, the detection apparatus is configured to perform and transmit measurements periodically, for example at a frequency which is parameterizable or at a frequency which is dependent on an activity state detected by the apparatus. The apparatus may, for example, comprise a gyroscopic module and/or an accelerometer for detecting the activity state of the subject. It is possible accordingly to detect the activity state of the subject during the sweat analyses, so as to facilitate analysis of the correlations between the activity state of the subject and the production of the chemical species analyzed.

According to one embodiment, the apparatus comprises a geolocation module.

According to one embodiment, the detection apparatus further comprises a communication device configured to transmit one or more measurement signals produced by the detection apparatus to an apparatus for storage or post-processing.

According to a second subject, the invention relates to a portable device comprising an above-described detection apparatus, the portable device being implemented in the form of: a watch, a telephone, a fabric, a headband, a garment or an undergarment.

According to one embodiment, the measurements produced by the detection apparatus are received, read and analyzed via a connected watch and/or a smartphone. The measurements may be received by wired, infrared, Bluetooth, Wi-Fi or 3G, 4G or 5G wave connection.

According to a third subject, the invention relates to a method for determining the production of at least nitric oxide dissolved in sweat by a human or animal subject, the method comprising:
selecting an investigation zone of an epidermis of said subject, applying an aforesaid detection apparatus for a duration necessary to produce the signal that is representative of a concentration of the nitric oxide NO dissolved in the flow of sweat and the signal that is representative of a flow rate of the flow of sweat, and
determining a measurement of the production of nitric oxide NO by the subject from the signal that is representative of the concentration of nitric oxide NO dissolved in the flow of sweat.

According to one embodiment, the method comprises: disinfecting the investigation zone beforehand.

By virtue of these characteristics, the measurement of nitric oxide in sweat is made more precise, since it does not include the manufacture of nitric oxide by bacteria and/or viruses that are present on the subject's skin. In other words, this ensures that the chemical compound detected comes from the sweat produced by the investigation zone, and not from a flow from the outside.

The measurements of the production of one or more of the aforesaid chemical species by the subject may be utilized in a variety of applications, for example for evaluating distress to the subject's vascular tissues on the basis of these measurements, or for evaluating the subject's cardiovascular capacity on the basis of these measurements.

Other possible applications are diagnostics, medical care and monitoring of diseases such as cardiovascular disease, neurodegenerative disease, pulmonary arterial hypertension, cancer, hypercholesterolemia, diabetes, systemic endothelial dysfunction, arteriosclerosis, thrombotic or ischemic disease, platelet accumulation inhibition dysfunction or leukocyte adhesion deficiency or cell proliferation dysfunction in smooth muscle fiber cells, bronchial inflammation, asthma, and Alzheimer's disease.

Other possible applications are the monitoring of growth and/or muscular distress in an individual, as for example an individual who is undergoing physical training, the prevention of injuries due to overtraining and/or the enhancement of the subject's muscle performance.

### BRIEF DESCRIPTION OF THE FIGURES

For better understanding of the subject matter of the invention, embodiments thereof as shown in the accompanying drawings will be described below, by way of purely illustrative and nonlimiting example. In these drawings:

FIG. 1 represents a schematic view of a subject seen from the back on whom a detection apparatus according to one embodiment has been placed,

FIG. 2 is a perspective view partially representing a multilayer structure for a detection apparatus according to one embodiment,

FIG. 3 represents a view in section, along the line II-II of the figure, of the multilayer structure,

FIG. 4 is an expanded view of the multilayer structure according to one embodiment,

FIG. 5 is an expanded view of the multilayer structure according to another embodiment,

FIG. 6 is an enlarged perspective view of an electrochemical sensor of the multilayer structure according to one embodiment,

FIG. 7 is an expanded view of the multilayer structure according to another embodiment,

FIG. 8 is a functional schematic representation of a microfluidic circuit which can be used in a detection apparatus,

FIG. 9 is another functional schematic representation of another microfluidic circuit which can be used in a detection apparatus,

FIG. 10 is another functional schematic representation of another microfluidic circuit which can be used in a detection apparatus,

FIG. 11 is a schematic perspective representation of an electrochemical sensor which can be used in the microfluidic circuit of FIGS. 2 to 10,

FIG. 12 is a chronogram illustrating a detection method which can be carried out with the electrochemical sensor of FIG. 11.

FIG. 13 (A) represents a scheme of an electrochemical sensor which can be used in the microfluidic circuit of FIGS. 2 to 10 of the detection apparatus according to one embodiment, and FIG. 13 (B) represents a method for detecting a sweat flow rate that may be carried out with the electrochemical sensor of FIG. 13 (A),

FIG. 14 represents a scheme of an electrochemical sensor of the detection apparatus according to one embodiment with the depletion function,

FIG. 15 represents an explanatory scheme for the depletion function according to one embodiment,

FIG. 16 is a chronogram illustrating a detection method which may be carried out with the electrochemical sensor of FIG. 14,

FIG. 17 illustrates schematically an electrochemical sensor according to an embodiment with five electrodes,

FIG. 18 illustrates schematically an electrochemical sensor according to an embodiment with six electrodes,

FIG. 19 illustrates schematically an embodiment of the multilayer structure further comprising a colorimetric detection device,

FIG. 20 is an expanded view of the multilayer structure according to another embodiment with a plurality of channels,

FIG. 21 is a functional schematic representation of a detection apparatus which can be used in the apparatus of FIG. 1,

FIG. 22 is a diagram of steps illustrating a method which may be implemented with the apparatus of FIG. 1,

FIG. 23 is a graph illustrating a result of the measurements which may be obtained with the apparatus of FIG. 1.

### DESCRIPTION OF THE EMBODIMENTS

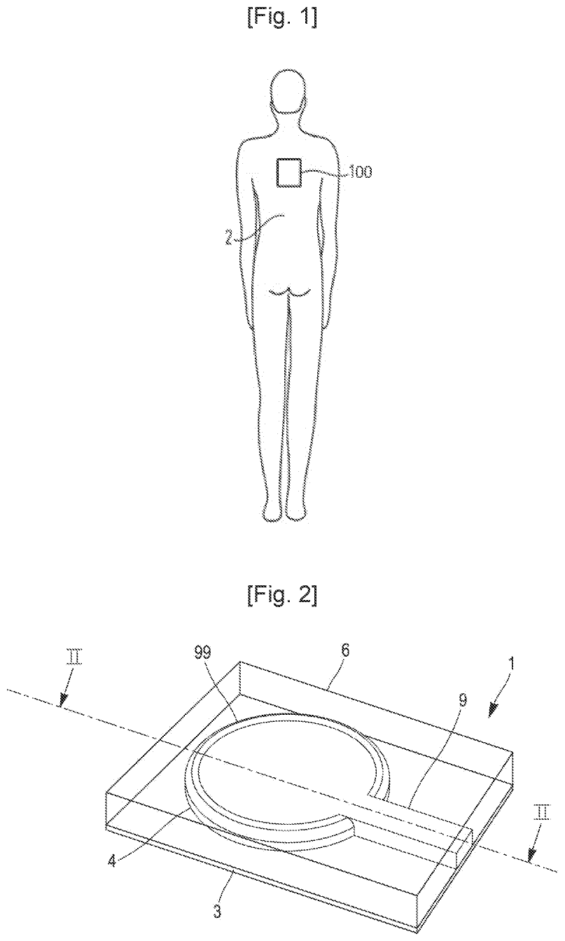

FIG. 1 represents a detection apparatus 100 disposed on the skin of a human subject 2, for example on the back of the subject, which is intended for performing quantitative measurements of chemical species dissolved in sweat, including nitric oxide dissolved in sweat, and optionally the nitrite ion or hydrogen peroxide. The detection apparatus 100 may be disposed on another part of the body, for example the nape of the neck, the shoulder, the arm or the leg.

The detection apparatus 100 comprises a microfluidic part and other functional parts which will be described further below, in particular a control device 40 (FIG. 21).

With reference to FIG. 2, the multilayer structure 1 takes the form, for example, of a low-profile casing which comprises a lower layer 3 made of a flexible and biocompatible material, preferably self-adhesive, which may be positioned directly on the skin of the subject, and a second layer 6 which lies atop the lower layer 3. The multilayer structure 1 is made from watertight materials, for example from polymer.

The second layer 6 is hollowed out in its thickness to form a microfluidic channel 9 and a sampling dome 99 situated over an opening 4 formed in the lower layer 3.

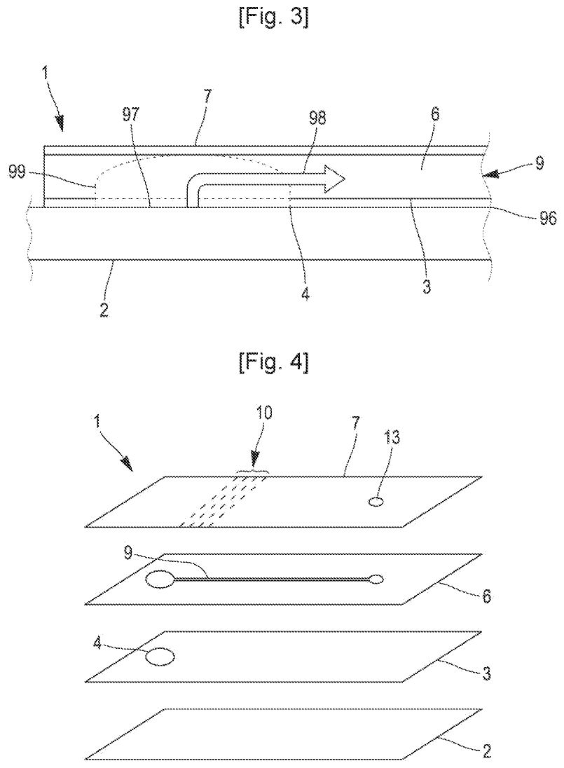

With reference to FIG. 3, the lower layer 3 is stuck to the skin 2 by an adhesive layer 96. A central part of the lower layer 3 and of the adhesive layer 96 comprises the circular opening 4 delimiting an investigation zone 97 on the skin 2 of the subject, being for example a few mm to a few cm in diameter. The circular opening 4 may adopt another form, for example, an ellipse, a triangle, a rectangle, a square, a polygon or some other form. The circular opening 4 is an entry orifice allowing a flow of sweat 98 to be conducted to, in particular for bringing the sweat into, the microfluidic channel 9. The flow of sweat 98 passes from the skin 2 of the subject to the microfluidic channel 9, passing across the circular opening 4.

In the embodiment of FIG. 3, an upper layer 7 covers the second layer 6 to form the microfluidic circuit at the top. The microfluidic circuit may therefore be formed through the whole thickness of the second layer 6, thereby making it easier to manufacture, for example by cutting or engraving.

A hydrophilic collector element (not shown), for example a fibrous body, such as cotton or a non-woven material, may be disposed in the circular opening 4 and the dome 99. The function of the collector element is to bring the sweat produced in the investigation zone to the microfluidic circuit.

With reference to FIG. 4, the multilayer structure comprises:
a lower layer 3 comprising an entry orifice 4 allowing passage of sweat,
an upper layer 7 comprising an outlet orifice 13,
a middle layer 6 situated between the lower layer 3 and the upper layer 7, the microfluidic circuit being formed in the thickness of the at least one middle layer 6 and extending parallel to the lower layer 3.

The microfluidic circuit consists of a microfluidic channel 9 which is in communication with the entry orifice 4 at a first end and in communication with the outlet orifice 13 at a second end. Accordingly, the flow of sweat from the skin 2 of the subject is conducted in the microfluidic channel 9, which conducts the sweat from the entry orifice 4 to the outlet orifice 13 by capillary action.

An electrochemical sensor 10 comprises four electrodes disposed on the inner face of the upper layer 7 closing the microfluidic channel at the top. The electrodes are therefore situated in the internal space of the microfluidic channel.

As a dimensional example, the entry orifice has a diameter of between 1 mm and 15 mm, the microfluidic channel has a length of between 0.5 cm and 5 cm and a width of between 25 μm and 500 μm, the middle layer has a thickness of between 10 μm and 200 μm, and the layers of the multilayer structure have a width of between 1 cm and 5 cm and a length of between 2 cm and 15 cm.

For example, the entry orifice 4 has a diameter of 5 mm, the microfluidic channel 9 has a length of 1.8 cm and a width of 100 μm, the middle layer has a thickness of less than 70 μm, for example 20 μm, and the layers of the multilayer structure have a width of 3 cm and a length of 9 cm.

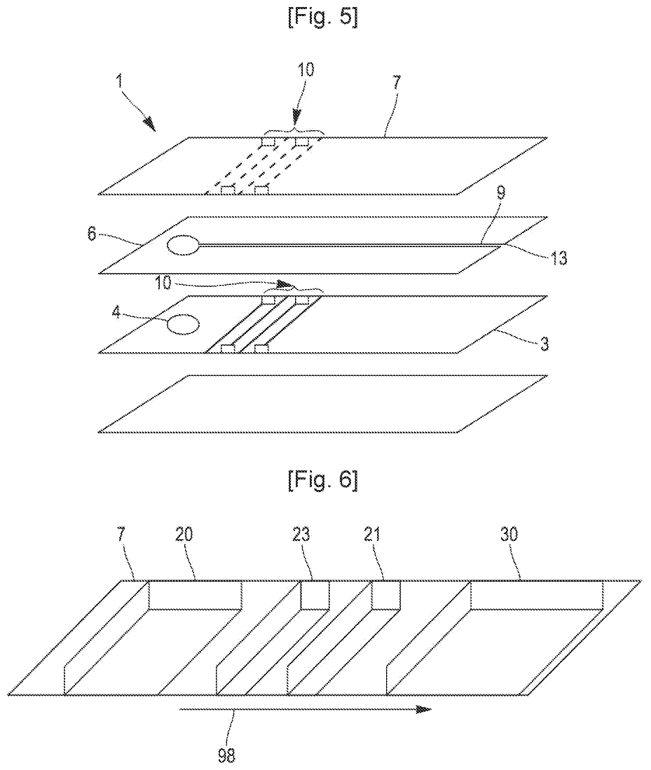

With reference to FIG. 5, the multilayer structure is similar to FIG. 4. However, in this embodiment, the outlet orifice 13 is situated in the middle layer 6, at one end of the middle layer. The detection apparatus 100 comprises an electrochemical sensor 10 comprising respectively four electrodes; each electrode comprises respectively two parts disposed facing one another, a first part disposed on an inner face of the upper layer 7 closing the microfluidic channel 9 at the top, and a second part disposed on an upper face of the lower layer 3 closing the microfluidic channel at the bottom. Each electrode part comprises a connector illustrated by a black rectangle, via which the electrodes may be electrically connected.

According to one embodiment, not illustrated, the detection apparatus may comprise a single electrochemical sensor 10 comprising four electrodes disposed on an upper face of the lower layer 3 closing the microfluidic channel 9 at the bottom.

FIG. 6 illustrates four electrodes arranged in a microfluidic channel, which may be the microfluidic channel 9 presented in FIGS. 4 and 5, for example. The four electrodes are applied metals disposed on the inner face of the upper layer 7 closing the microfluidic channel 9 at the top. The four electrodes are disposed successively in a longitudinal direction of the microfluidic channel and comprise: a working electrode 20, implemented here in the form of a depletion electrode which operates in a manner that will be detailed further below, a second working electrode 23, a reference electrode 21, and a counter-electrode 30. The height of the electrodes is between 1 and 50 nanometers (nm), the space between the electrodes is between 10 and 10 000 micrometers (μm), and the width of the electrodes is between 1 and 1000 μm. The electrodes may be manufactured, for example, of platinum (Pt), of gold (Au), of silver (Ag), or of silver chloride (AgCl).

At least one of the electrodes may also be covered entirely or partly with polyeugenol. platinum black or polyphenol. The electrodes are configured to perform one or more of these actions: depleting, measuring the concentration of nitric oxide, measuring the concentration of at least one other chemical component, and measuring the flow rate of the flow of sweat flowing in the microfluidic channel 9.

FIG. 7 illustrates the multilayer structure 1 similar to FIG. 4, in which a second, sealing middle layer 26 is situated between the first middle layer 6 and the upper layer 7, and the second, sealing middle layer 26 comprises an opening 27 at the electrodes, allowing the electrodes to be contacted with the flow of sweat circulating in the microfluidic channel 9. The second, sealing middle layer 26 further comprises an intermediate opening 28 which communicates with the outlet orifice 13 of the upper layer 7 so as to enable discharge of sweat. For example, the opening 27 is rectangular in form and has a length of 5 mm and a width of 200 μm.

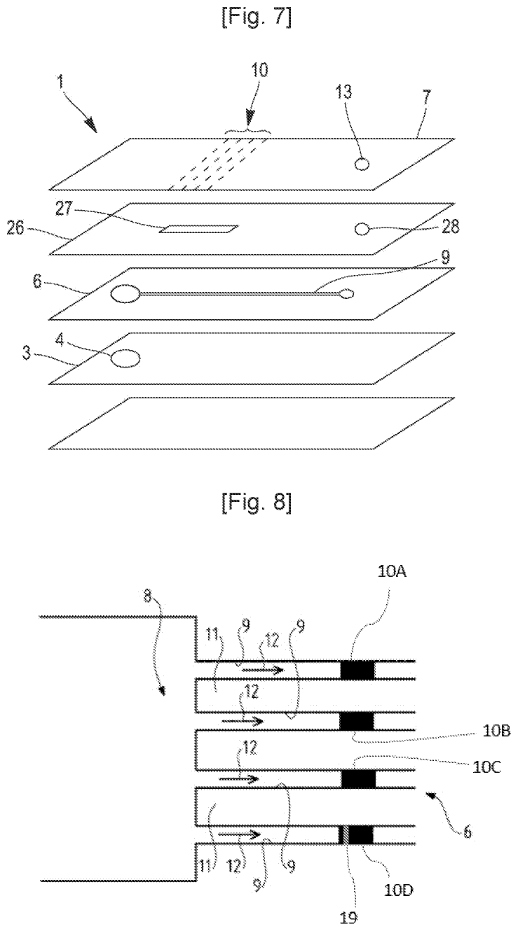

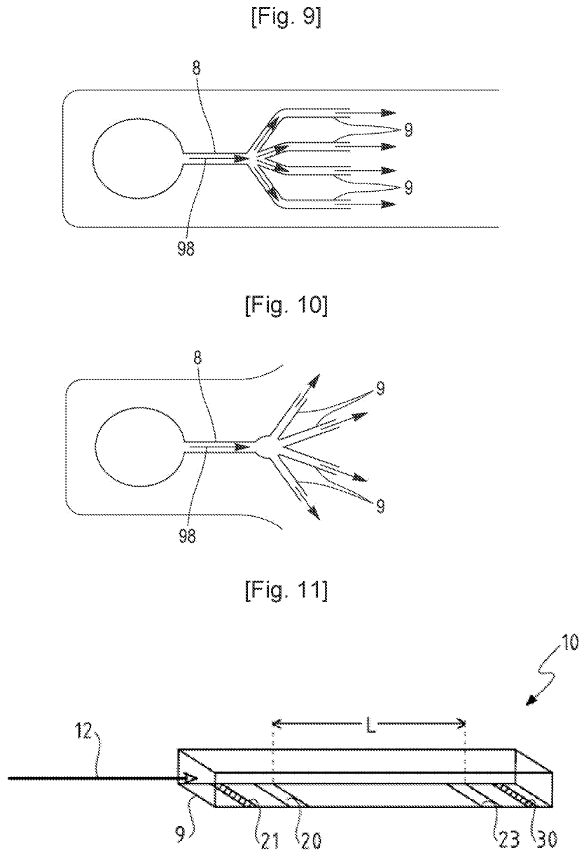

With reference to FIG. 8, the middle layer 6 comprises a microfluidic circuit 8 fed with a flow of sweat. The sweat is received from the entry orifice of the lower layer toward the microfluidic circuit 8. The microfluidic circuit 8 may comprise one or more microfluidic channels 9, specifically four parallel microfluidic channels 9 in the example represented. However, the microfluidic circuit 8 may adopt different forms; FIG. 9, for example, represents a microfluidic circuit 8 comprising parallel microfluidic channels 9, and FIG. 10 represents four microfluidic channels 9 distributed radially. The microfluidic channels 9 are formed, for example, in the thickness of the middle layer 6 and are separated by partitions 11. Each microfluidic channel 9 is respectively separated from the other microfluidic channels, within which the sweat may circulate independently. The number of microfluidic channels 9 may be higher or lower than in these figures.

Each fluidic circuit 9 is equipped with a sensor 10A, 10B, 10C or 10D. The arrows 12 illustrate the direction of flow of sweat in the microfluidic channels 9. Via the outlet orifice 13, the microfluidic channels 9 end preferably in a drainage reservoir which retains the analyzed fluids, so as to prevent the reaction products of the electrolysis coming back into contact with the subject's skin.

The sensors 10A, 10B, 10C and 10D arranged in the microfluidic circuits 9 for analyzing the sweat are preferably electrochemical sensors. The operating principle of an electrochemical sensor is that of wholly or partially electrolyzing the solution present in the fluidic channel 9 between a working electrode and a counter-electrode. An electrochemical sensor of this kind may be implemented in a variety of ways, in particular in miniaturized form with dimensions of the order of a millimeter.

A description will now be given of a number of embodiment examples of the electrochemical sensors, with reference to FIG. 8.

### EXAMPLES

### Example 1

The sensor 10A is intended for detecting hydrogen peroxide. It therefore operates with a potential difference equal to the oxidation potential of hydrogen peroxide, E_{H}_{₂}_{O}_{₂}. The sensor 10B is intended for detecting nitric oxide. It therefore operates with a potential difference equal to the oxidation potential of nitric oxide, E_{NO}. The sensor 10C is intended for detecting the nitrite ion. It therefore operates with a potential difference equal to the oxidation potential of the nitrite ion, E_{NO}_{_{2−}}.

The sensors 10A, 10B, 10C carry out synchronous measurements of the instantaneous intensities, denoted i_{oxdn}, of the faradaic currents linked to the electrochemical oxidation of the aforesaid chemical species. The sensors 10A, 10B, 10C therefore allow the detection and quantification of the instantaneous concentration of the aforesaid chemical species.

Each of the three aforesaid chemical species can be detected by amperometric measurements with the aid of microelectrodes. The latter consist, for example, of strips of platinum covered with a thin layer, for example of micrometric dimensions, of platinum black applied by electrochemical reduction, in an aqueous medium, of the anion of a platinum salt, Pt(Cl)₆⁴⁻.

The three chemical species (NO, NO₂⁻ and H₂O₂) can be distinguished by the fact that their oxidation potentials on these electrodes are clearly separated, occurring in the following order: E_{H2O2}<E_{NO}<E_{NO2-}. However, the faradaic currents are additive. The measured current at the oxidation potential of each chemical species therefore adds to the elementary currents linked to the oxidation of this chemical species and to the oxidation of all of the chemical species which have lower oxidation potentials.

Thus, only the species H₂O₂ can be oxidized at the oxidation potential EH₂O₂. The species H₂O₂ and NO can be oxidized at the oxidation potential E_{NO}. The three species can be oxidized at the oxidation potential E_{NO2-}. The currents measured by the sensors 10A to 10C, respectively denoted iₒₓₒₙ (EH₂O₂), iₒₓₒₙ(E_{NO}) and iₒₓₒₙ (E_{NO2-}), therefore satisfy the following equations:

$${i}_{oxdn} ( {E}_{H 2 O 2} ) = a 1 {i}_{H 2 O 2}$$

$${i}_{oxdn} ( {E}_{NO} ) = a 2 {i}_{H 2 O 2} + a 3 {i}_{NO}$$

$${i}_{oxdn} ( {E}_{NO {2}^{-}} ) = a 4 {i}_{H 2 O 2} + a 5 {i}_{NO} + a 6 {i}_{NO {2}^{-}}$$

in which the coefficients a1 to a6 represent calibration constants for the sensors, which can be measured experimentally.

Accordingly, via subtractions which are easily implemented on an electronic circuit, the following are obtained:

$${i}_{{H}_{2} {O}_{2}} = ( 1 / a 1 ) {i}_{oxdn} ( {E}_{{H}_{2} {O}_{2}} )$$

$${i}_{NO} = ( 1 / a 3 ) {i}_{oxdn} ( {E}_{NO} ) - ( a 2 / a 1 ) \cdot ( 1 / a 3 ) {i}_{oxdn} ( {E}_{{H}_{2} {O}_{2}} )$$

$${i}_{{NO}_{{2}^{-}}} = ( 1 / a 6 ) {i}_{oxdn} ( {E}_{{NO}_{{2}^{-}}} ) - ( a 4 / a 6 ) {i}_{{H}_{2} {O}_{2}} - ( a 5 / a 6 ) {i}_{NO}$$

At any time t, the instantaneous intensity of the faradaic oxidation current, iₛ(t), for each chemical species S is proportional to its concentration, Cₛ(f), in the volume of fluid situated above the electrodes which detect it. The proportionality factor depends on a form factor, denoted γ, which is a function of the geometry of the sensor, and on the Faraday constant, denoted nₛ, consumed per mole of the chemical species, i.e.:

$${n}_{H 2 O 2} = {n}_{NO {2}^{-}} = 2 and {n}_{NO} = 1$$

It will be recalled that F denotes the faraday, i.e. 96 500 coulombs, the value for the charge of one mole of electrons.

The form factor γ is a constant factor imposed by the geometry of the electrochemical sensor, which can be evaluated theoretically and measured experimentally by calibration. For the sake of simplicity, the three sensors 10A to 10C are considered below to have identical geometries, such that the form factor γ is the same for all of the sensors.

The result is that the concentrations of the chemical species can be obtained from the currents measured by the sensors 10A to 10C, with the aid of the following expressions, in which the temporal variable t has been specified:

$${C}_{{H}_{2} {O}_{2}} ( t ) = {i}_{oxdn} ( {E}_{{H}_{2} {O}_{2}} , t ) / ( 2 F γ )$$

$${C}_{NO} ( t ) = [ {i}_{oxdn} ( {E}_{NO} , t ) - {i}_{oxdn} ( {E}_{{H}_{2} {O}_{2}} , t ) ] / ( F γ )$$

$${C}_{{NO}_{{2}^{-}}} ( t ) = [ {i}_{oxdn} ( {E}_{{NO}_{{2}^{-}}} , t ) - {i}_{oxdn} ( {E}_{NO} , t ) ] / ( 2 F γ )$$

In example 1, the three sensors 10A to 10C can therefore operate in parallel, each with a constant oxidation potential, namely E_{H2O2}, E_{NO} and E_{NO2-} respectively.

In a variant embodiment, only NO and NO₂⁻ are detected. This embodiment is particularly advantageous when the measurement of H₂O₂ is not significant and does not influence the results of the intended objective. The concentration C_{H2O2}(t) presented above is then considered to be uniformly zero, i.e. C_{H2O2}(t)=0. The system of equations is therefore simplified.

### Example 2

In example 2, a single microfluidic channel 9 and a single sensor 10A are used; the others being able to be omitted.

In this case, the sensor 10A operates sequentially in order to detect the aforesaid chemical species during three successive steps. The oxidation potential is therefore switched between three potential stages, respectively equal to the three oxidation potentials mentioned above, for example periodically in accordance with the sequence E_{H2O2}→E_{NO}→E_{NO2-}→E_{H2O2}→E_{NO}→E_{NO2-}→ etc.

In this case, each oxidation potential is maintained for a duration that is very long compared with the time constant for the working electrode, this time constant being, for example, a few milliseconds for the microelectrodes employed in the microfluidic channels, and measurements of the current are carried out at the end of each constant potential stage.

The remaining measurement signals can be processed using the same equations as in example 1.

### Example 3

Because nitric oxide is a small molecule that is both hydrophilic and lipophilic, it can easily pass through thin layers of organic polymer, in contrast to the other two species H₂O₂ and NO₂⁻. Thus, it can be detected in isolation with the aid of an electrochemical sensor protected by a layer of this type, for example with a working electrode made of platinized platinum coated with a thin layer of polyeugenol (4-allyl-2-methoxyphenol) applied by electropolymerization.

In example 3, the working electrode of the sensor 10D is therefore coated by the layer which is schematically represented by the numeral 19. The instantaneous concentration of nitric oxide can therefore be measured independently of that of the chemical species H₂O₂ and NO₂⁻, in accordance with the expression:

$${C}_{NO} ( t ) = {[ {i}_{oxdn} ( {E}_{NO} , t ) ]}_{eugenol} / ( F γ ) .$$

Here, i_{oxdn}(E_{NO}, t)]_{eugenol} denotes the current measured by the sensor 10D.

The other sensors 10A to 10C and the other microfluidic channels 9 can be omitted. This method can therefore advantageously be used with a single sensor when only the concentration of NO is desired.

### Example 4

In this case, the sensor 10D of example 3 is amalgamated with the sensors 10A to 10C of example 1 or with the sensor 10A of example 2. This configuration can be used to obtain two measurements that are independent of the concentration of dissolved nitric oxide, and thus to check the consistency of the measurements, in particular by verifying that the sensors do not exhibit drift, which is linked for example to partial deactivation of the surface of one of the electrodes.

In this case, the electrochemical electronic control device 40 (FIG. 21) is preferably configured to compare the two measurements of the concentration of nitric oxide and to emit an alarm when the result of the comparison satisfies a predefined criterion, for example if it exceeds a predefined threshold.

In examples 1 to 4 above, the measured instantaneous faradaic currents can be used to measure the concentration of the chemical species in the analyzed solution. As a consequence, in a static system, the intensity of the current is sufficient to document the production of the detected species.

However, when the detection apparatus 100 is applied to an essentially dynamic physiological system, it is desirable also to be able to have quantitative access to the dynamics of production of each chemical species by the cardiovascular system, for example during exertion tests or during medical monitoring. Under dynamic conditions, in order to access the instantaneous quantity of a chemical species, denoted ΔQ(t), produced over a short period of time, denoted Δt(t), it is desirable to know the mean concentration, Cₛ(t), of the chemical species and the volume flow rate of the analyzed fluid simultaneously, namely:

$$d ( t ) = ( Δ V / Δ f )$$

in which ΔV designates the volume scanned during the time interval Δt. Thus, the intensity of the production flow, denoted Pₛ(t), of a chemical species S at a time t is given by:

$${P}_{s} ( t ) = [ Δ Q / Δ t ] ( t ) = {C}_{s} ( t ) \cdot d ( t )$$

in which the mean concentration Cₛ(t) is obtained from the mean intensities of the electrochemical oxidation currents measured between the times t and t+Δt.

In the context of the dynamic applications envisaged, it is therefore desirable for the detection apparatus 100 to measure, at the same time and at each time t required by the desired accuracy for monitoring the physiological status of the patient over time, for example once per minute, the mean intensities, iₐᵥ(f), of the faradaic current linked to the electrochemical oxidation of the one or more chemical species being monitored and the value for the volume flow rate d(t) of sweat at the time t in the corresponding fluidic circuit.

FIG. 11 illustrates an embodiment of an electrochemical sensor 10 that can meet this dual requirement in an integrated manner. This electrochemical sensor 10 comprises at least one pair of working electrodes 20, 23. A strip microelectrode of this type may be produced from platinized (platinum black) platinum, which may or may not be covered with a layer of electropolymerized eugenol of micrometric dimensions. A strip microelectrode of this type may be implanted by microfabrication, for example by CVD and/or lithography. This strip microelectrode or these strip microelectrodes can be used to electrochemically oxidize the selected chemical species.

The microfluidic circuit 9 of FIG. 11 is further equipped with a reference electrode 21 that is produced, for example, in the form of an Ag/AgCl microstrip and placed upstream of the pair of working electrodes 20, 23. Finally, the fluidic circuit 9 is equipped with a counter-electrode 30 made of platinized platinum and placed downstream of the pair of working electrodes 20, 23. Notwithstanding the functional schematic representation of FIG. 11, the surface area of the counter-electrode 30 is in fact two to three times larger than that of the other electrodes.

The assembly of the microfluidic channel 9 with the electrodes 20, 21, 23, 30 is bathed in a lamina of sweat, not shown, and thus constitutes a microfluidic electrochemical cell with four electrodes. Each of the electrodes 20, 21, 23, 30 is connected to an electrochemical electronic control device 40 (FIG. 21) by means of electrical contacts insulated from the sweat.

This embodiment of an electrochemical sensor 10 may be employed in one or more of the aforementioned microfluidic circuits 9.

In order to measure the volume flow rate d(t), the electrochemical sensor 10 has to include the pair of working electrodes 20, 23. The solution described here is simple and readily industrializable, because it has no moving parts and it makes no claim to be hydrodynamic. It does not require any intervention aimed at modulating the flow rate of the fluid, while being suitable for any reasonable physiological flow rate.

The two working electrodes 20, 23, for example two strips of platinized platinum, may act as working microelectrodes, are electrically independent and are spaced apart by a distance L along the path of the fluid analyzed in the microfluidic circuit 9. The two working electrodes 20 and 23 are, for example, installed on the bottom of a linear channel, the section of which has a constant area A.

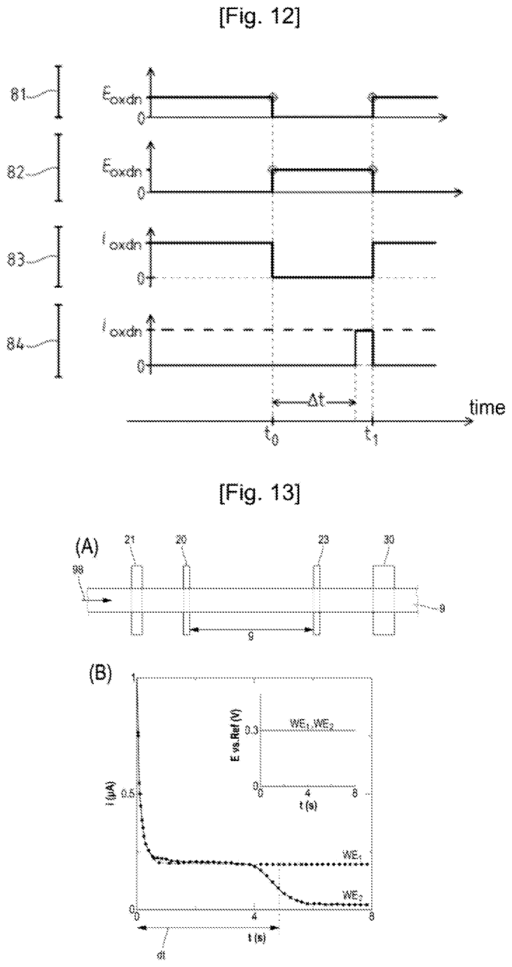

The working electrode 23 positioned downstream is used in accordance with the method illustrated in FIG. 12, which comprises two steps. The graph 81 represents the electrical potential applied to the working electrode 20 as a function of time. The graph 82 represents the electrical potential applied to the working electrode 23 as a function of time. The potentials indicated as “0” on graphs 81 and 82 in fact signify disconnection of the corresponding electrode (open circuit). The graph 83 represents the faradaic current measured at the working electrode 20 as a function of time. The graph 84 represents the faradaic current measured at the working electrode 23 as a function of time.

During a first step carried out over a range of time prior to the time to, the potential E_{oxdn} applied to the working electrode 20 is sufficient to allow oxidation of the one or more target chemical species, while the downstream working electrode 23 is disconnected. The working electrode 20 positioned upstream can then be used to continuously record the instantaneous electrochemical current, i_{oxdn}(f), which, following any calculations indicated further above, then indicates the concentration C(f) of the one or more target chemical species in the fluid analyzed.

During a second step carried out over a range of time from the time to, the working electrode 20 is disconnected and the potential E_{oxdn} is applied to the downstream working electrode 23.

At the time to, the flow of sweat passing above the working electrode 23 has already been electrolyzed (completely or partially) during its passage above the working electrode 20 which is located upstream, in a manner such that the concentration of the target chemical species there is zero, or at least much lower than before it entered the electrochemical sensor. The intensity i_{oxdn} of the current detected by the working electrode 23 (graph 84) is therefore zero (or at least much lower than that of the current i_{oxdn} detected at the working electrode 20 before the time t₀).

At the time t₀+Δt, the working electrode 23 starts to analyze a non-electrolyzed solution and the current intensity i_{oxdn} that it detects becomes of the same order as that detected by the working electrode 20 before the time t₀. The growth in the current, schematized by a step in FIG. 12, is detected by an ad-hoc electronic circuit. The duration Δt, which is the delay between this growth and the moment t0 of disconnection of the working electrode 20, represents the time necessary for the flow of sweat to transit between the two working electrodes 20 and 23. The duration Δt is represented by a double-headed arrow at the bottom of FIG. 12. In order to simplify the representation, it has been assumed in FIG. 12 that electrolysis of the target chemical species is complete when the working electrode 20 is connected. The same measurement principles are applicable when this electrolysis is only partial.

The rate of flow v(t) and the flow rate d(t) may therefore be estimated as follows:

$$v ( t ) = L / Δ t$$

$$d ( t ) = A \cdot v ( t )$$

The potential E_{oxdn} applied to the working electrode 23 is sufficient to enable oxidation of the one or more target chemical species, while the working electrode 20 is disconnected.

The measurement of concentration may therefore optionally be continued for a certain period with the working electrode 23. The second step ends with the disconnection of the working electrode 23 at the time t₁. The working electrode 20 may then be reconnected and the method can be repeated as many times as necessary in order to evaluate the flow rate d(t) at successive times.

The distance L between the two working electrodes 20 and 23 is preferably sufficiently small, for example of the order of 1 mm, for the changes in the physiological response of the patient to be negligible over the period Δt.

A second method of measuring the volume flow rate of the sweat flow is illustrated in FIG. 13. In contrast to the explanation above, the measurement principle here is that of detecting a drop in the measurement current of a chemical compound, for example nitric oxide. FIG. 13A illustrates schematically an assembly of four electrodes disposed in a microfluidic channel 9 according to one embodiment. The electrodes are disposed successively in a longitudinal direction of the microfluidic channel 9. The electrodes are disposed as follows: a reference electrode 21 disposed upstream and receiving the flow of sweat 98 first; a first and a second working electrode 20, 23, spaced apart by a distance g represented by the double-headed arrow; and a counter-electrode 30. The measurement principle is illustrated in FIG. 13 (B) and comprises the connection and the polarization of the two working electrodes 20, 23 simultaneously, then the monitoring over time of the change in the current of the second working electrode 23 situated downstream in the microfluidic channel 9. From the start of the polarization (at t=0), the two working electrodes 20, 23 oxidize the same species. The first, upstream working electrode 20 brings about depletion of these species and, after a duration dt, induces a decrease in the current of the second working electrode 23, situated downstream. dt is the time required for the depletion zone comprising the species oxidized by the first working electrode 20 to reach the second electrode 23, by convection. It is observed, therefore, that after the simultaneous polarization of the first 20 and the second 23 working electrode at t=0, the current of the second working electrode 23, situated downstream, reduces in the duration dt. By way of example, the duration dt in FIG. 13 (B) is estimated at five seconds. Accordingly, the linear rate v of the flow is obtained by the simple relation v=g/dt.

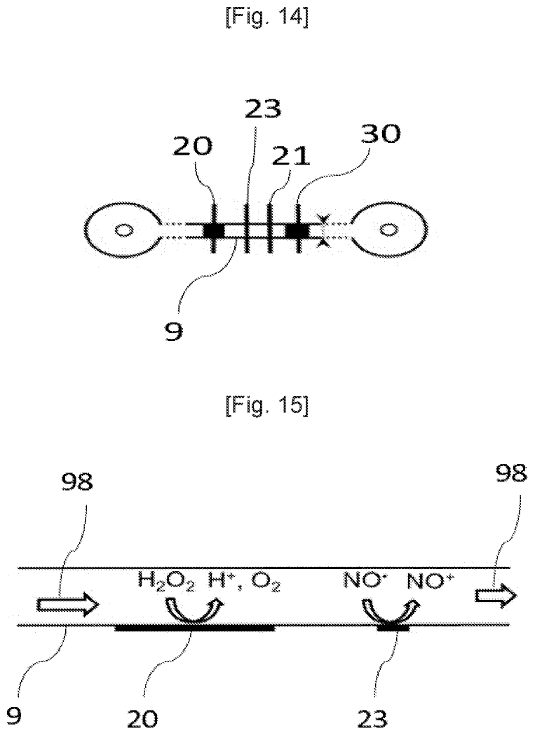

This principle may be exploited in combination with a depletion function described with reference to FIGS. 14 to 16 below.

FIG. 14 illustrates the microfluidic circuit comprising an electrochemical sensor comprising four electrodes disposed successively in a longitudinal direction of the microfluidic channel 9. A depletion electrode 20 is placed upstream of the working electrode 23, for the depletion of the interfering species for which downstream detection is unwanted. By polarization of the depletion electrode 20, the interfering chemical species are oxidized selectively. The depletion electrode 20 is wide, so as to optimize depletion and to remove virtually all of the one or more interfering species, for example to remove hydrogen peroxide H₂O₂. The chemical species of interest will be oxidized by the working electrode 20 situated downstream. A configuration of this kind may be used for depletion of one or more interfering chemical species having an oxidation potential lower than the oxidation potential of nitric oxide, and for improving measurement of a concentration of a chemical compound, nitric oxide for example. The counter-electrode 30 and the reference electrode 20 are necessary for controlling the potentials and for circulating currents in the measuring zone of the electrochemical sensor.

In the microfluidic channel 9 equipped with this depletion electrode 20, a concentration of nitric oxide can be obtained directly, with no need to resolve the system of linear equations presented further above.

In a variant embodiment in which it is desired to obtain the measurement of the concentration of NO₂⁻ in the microfluidic channel 9, the depletion electrode 20 may be configured for removing nitric oxide.

FIG. 15 presents an example of depletion of hydrogen peroxide with the sensor of FIG. 14. The reference electrode and the counter-electrode are not shown. The depletion electrode 20 and the working electrode 23 are polarized independently of one another under constant sweat flow rate conditions. The direction of the flow of sweat is represented via the arrows in the microfluidic channel 9. The depletion electrode 20 is very large relative to the working electrode 23, for example eight times larger, so as to remove all of the interfering species upstream, here hydrogen peroxide (H₂O₂), by oxidation. The chemical species for which measurement of the concentration is desired, here nitric oxide, is not oxidized by the depletion electrode 20. The nitric oxide will be oxidized downstream by the working electrode 23. Accordingly, the depletion electrode 20 and the working electrode 23 are polarized to potentials such that E_{DE}<E_{WE}, so as to obtain the selectivity in the detection of nitric oxide.

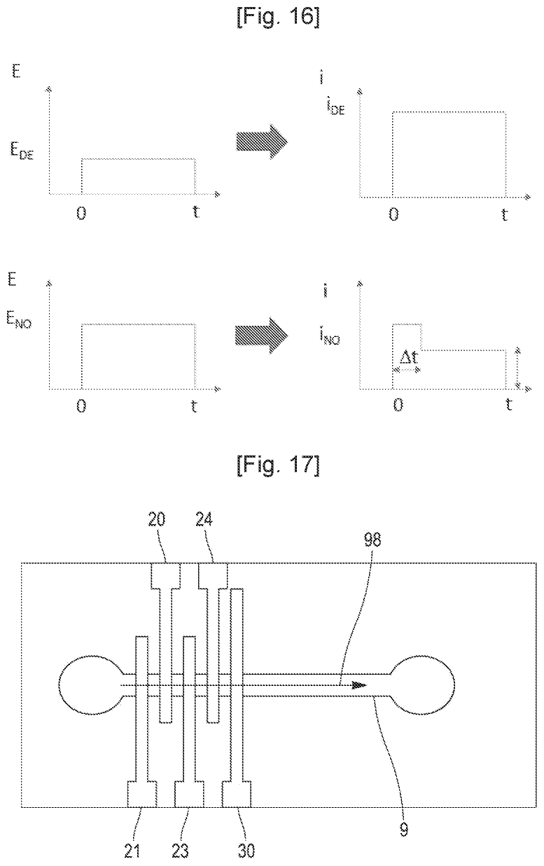

As set out above, the flow rate of the flow of sweat may be measured via the working electrode 20, which performs the depletion, and the working electrode 23, which oxidizes the nitric oxide, for example. FIG. 16 illustrates measurement of the flow rate of the flow of sweat 98 according to a method similar to that described in FIG. 13A and B. Accordingly, the depletion electrode 20, which oxidizes the chemical species whose detection is not desired, for example hydrogen peroxide, and the working electrode 23, which oxidizes nitric oxide, are connected and polarized simultaneously. In a first period, therefore, the working electrode 23 will detect nitric oxide NO and also hydrogen peroxide, as the volume of sweat situated between the depletion electrode 20 and the working electrode 23 will not be depleted by the depletion electrode 20. In a second period, the depletion electrode 20 brings about depletion of hydrogen peroxide, thereby, in a manner similar to that described with FIG. 13, after a duration dt, inducing a decrease in the current of the working electrode 23. In this embodiment, the current of the working electrode 23 reduces but does not become zero or virtually zero, because following the depletion, the working electrode 23 selectively detects the nitric oxide.

The methods for flow rate measurement described above may be employed simultaneously in all of the parallel microfluidic channels. However, if these channels are configured and fed in a similar manner, a single flow rate measurement may be sufficient. In that case, the flow rate measurement method described above may be employed in a single microfluidic channel 9. Furthermore, these flow rate measurement methods can be combined with the sensors of the various examples.

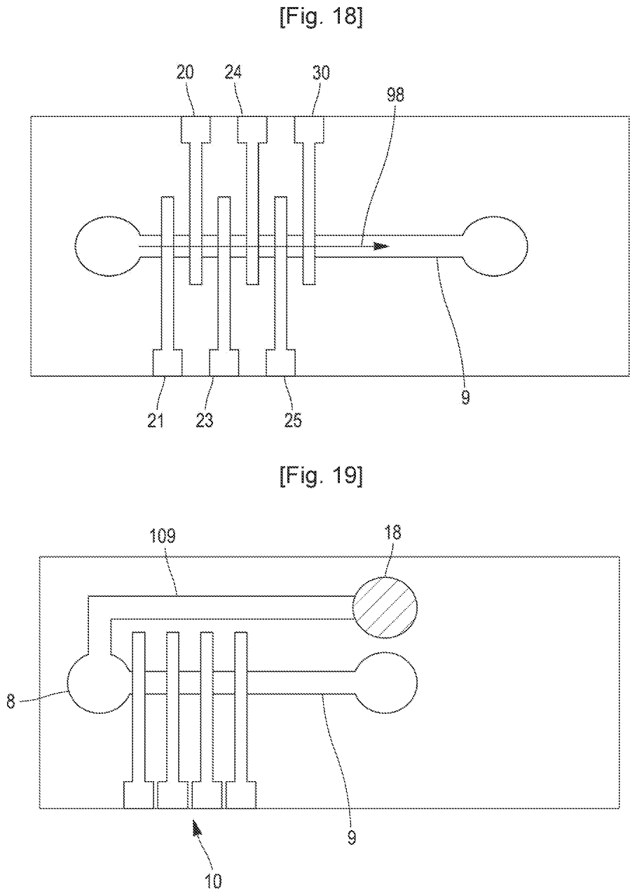

With reference to FIGS. 18 and 19, electrochemical sensors are described that employ a larger number of electrodes.

The electrodes presented in FIGS. 17 to 19 may be disposed on the inner face of the upper layer closing the microfluidic channel at the top and/or disposed on an upper face of the lower layer closing the microfluidic channel at the bottom. The schematic representation does not differentiate the different layers.

FIG. 17 represents an electrode configuration according to an embodiment in which the electrochemical sensor comprises, from left to right in FIG. 17, a reference electrode 21, a depletion electrode 20, a first working electrode 23, a second working electrode 24, and a counter-electrode 30. With this configuration it is possible, in the following order, to carry out:
depletion of an unwanted chemical species such as hydrogen peroxide via polarization of the depletion electrode 20 to the potential for oxidation of hydrogen peroxide; oxidation of nitric oxide via the first working electrode 23, with an oxidation potential greater than the potential for oxidation of hydrogen peroxide;
oxidation of nitrites via the second working electrode 24, with an oxidation potential greater than the potential for oxidation of hydrogen peroxide and than the potential for oxidation of nitric oxide, and measurement of the flow rate of the flow of sweat by a delay between the first working electrode 23 and the second working electrode 24. The flow measurement may also be measured by a delay between the depletion electrode 20 and the first working electrode 23.

FIG. 18 illustrates an embodiment similar to the preceding figure. The embodiment differs in that the electrochemical sensor comprises a third working electrode 25, situated between the second working electrode 24 and the counter-electrode 30. The flow rate of the flow of sweat 98 is measured between the second working electrode 24 and the third working electrode 25. By spacing the electrodes 24 and 25 apart, in other words by shifting the electrodes 25 and 30 further toward the end of the channel 9, it is possible to improve the resolution of the flow rate measurement for the flow of sweat 98.

FIG. 19 represents a variant embodiment in which the detection apparatus comprises a microfluidic circuit 8 comprising two parallel microfluidic channels 9 and 109. A first microfluidic channel 9 comprises an electrochemical sensor. The assembly of the electrochemical sensors disclosed here may be integrated in this first microfluidic channel 9. The second microfluidic channel 109 comprises a colorimetric detection device 18. The colorimetric detection device 18 comprises a hydrophilic microporous membrane. The hydrophilic microporous membrane comprises at least one chemical reagent capable of reacting with a chemical species for detection, for example, of: nitrite ion, hydrogen peroxide, peroxynitrite, sulfur dioxide, hydrogen sulfide, nitric oxide, carbon monoxide and hypochlorous acid. In proportion with the accumulation of the chemical species for detection in the porous body impregnated with the reagent, the reagent changes color, and the intensity of its specific color increases. The intensity of color may be detected to provide a quantitative measure of the quantity of the chemical species dissolved in the flow of sweat. For example, the reagent used is the Griess reagent, allowing detection of the nitrite ion by providing a red-colored indicator.

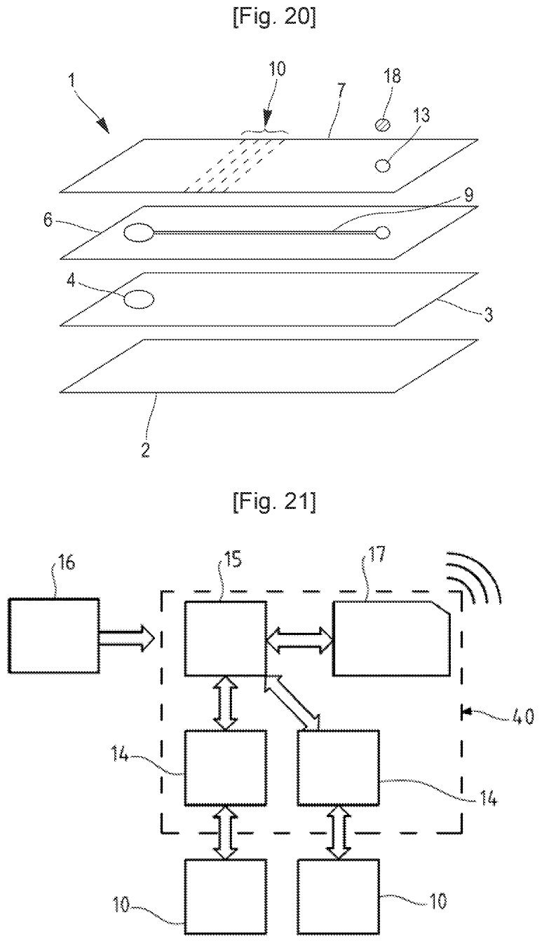

According to a variant embodiment presented in FIG. 20, the colorimetric detection device 18 is placed at the outlet of the microfluidic channel 9 in series with an electrochemical sensor 10. For this, FIG. 20 shows a multilayer structure 1 identical to FIG. 4. Since the colorimetric detection device 18 is disposed downstream of the electrochemical sensor, it receives a solution which has been electrolyzed by the electrochemical sensor 10. Colorimetric measurement is possible, however, with the proviso that the species for detection has not been substantially adversely affected by the operation of the electrochemical sensor 10.

The methods for detecting concentration and flow rate described above may be carried out in an automated manner with the aid of an electronic control device 40, which is preferably integrated into the detection apparatus 100.

With reference to FIG. 21, an embodiment of the electronic control device 40 which can be integrated into the detection apparatus 100, for example in the form of an electronic circuit board, is now described.

The or each electrochemical sensor 10 is connected to an analog-to-digital converter 14, which in turn supplies a processor 15. The processor 15 is, for example, programmed to execute the methods for detecting concentration and flow rate described above.

An energy source 16, for example a battery, supplies the electronic control device 40. A communication module 17, which may be wired or wireless, may also be provided in order to communicate the results of the measurements of concentration, flow rate and/or quantitative material flow, for one or each target chemical species, to a storage or post-processing apparatus.

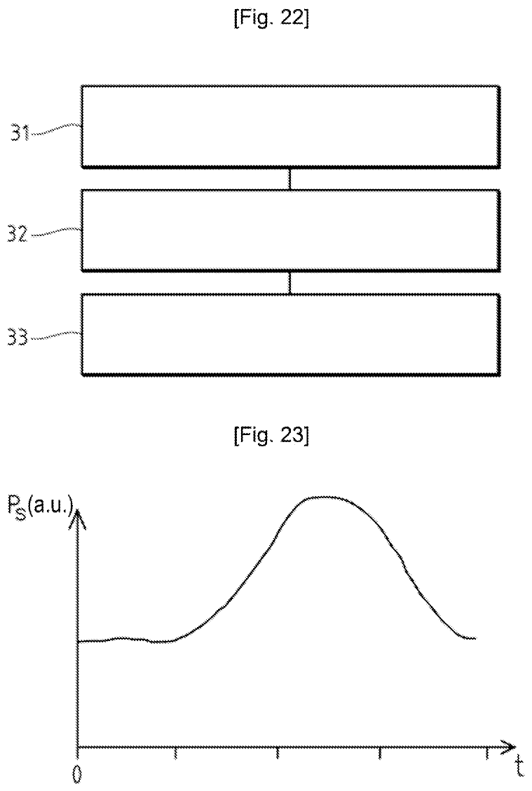

FIG. 22 represents a method which may be executed by the processor 15 in one embodiment.

In step 31, the instantaneous concentration Cs(t) of a chemical species S is determined from electrochemical measurements.

In step 32, the volume flow rate d(t) in the corresponding fluidic circuit is determined.

In step 33, the quantitative material flow for the chemical species under consideration is determined on the basis of Cs(t) and d(t), for example:

$${P}_{s} ( t ) = {C}_{s} ( t ) \cdot d ( t )$$

FIG. 23 is a graph illustrating a measurement signal for the quantitative material flow as a function of time that may be obtained with the detection apparatus 100, for example during an exertion test on a subject for the species NO.

The electronic control device 40 optionally comprises other functional modules, for example a gyroscopic module and/or an accelerometer module for detecting the orientation and movements of the subject and also the level of activity of the subject, and a temperature sensor in order to measure the temperature of the subject's epidermis. It is useful to know the temperature of the skin for the purposes of correlations between the temperature and the dilation of the vessels.

Certain elements of the detection apparatus 100, in particular the electronic control device 40, may be realized in different forms, in a unitary or distributed manner, using physical and/or software components. Physical components that may be used are application-specific integrated circuits (ASICs), field programmable gate arrays (FPGAs), or microprocessors. The software components may be written in various programming languages, for example C, C++, Java or VHDL. This list is not exhaustive.

Although the invention has been described in connection with a number of particular embodiments, it is clear that it is not in any way limited to them and that it encompasses all equivalent techniques for the means described and also combinations thereof if they fall within the scope of the invention. For example, the detection apparatuses described may comprise an additional microfluidic channel or different electrochemical sensors and/or sensors comprising a different number of electrodes.

The use of the verb “comprise”, “encompass” or “include” and the conjugated forms thereof does not exclude the presence of elements or steps other than those set out in a claim.

In the claims, none of the reference signs in parentheses should be interpreted as limiting the claim.

1. A detection apparatus for placement on an investigation zone (97) of an epidermis of a human or animal subject for detecting at least nitric oxide dissolved in sweat, said detection apparatus (100) comprising:
a structure defining a microfluidic circuit (8), the structure comprising an entry orifice (4) allowing passage of sweat from the epidermis, the microfluidic circuit (8) comprising at least one microfluidic channel (9) for guiding a flow of sweat (98), the microfluidic channel (9) being in communication with the entry orifice (4),
at least one electrochemical sensor (10) configured to produce at least one signal that is representative of a concentration of the nitric oxide dissolved in the flow of sweat (98) in the microfluidic channel (9),
wherein the electrochemical sensor (10) comprises at least four electrodes disposed successively in a longitudinal direction of the microfluidic channel (9), the at least four electrodes comprising a reference electrode (21), at least two working electrodes (20, 23) and a counter-electrode (30), and
wherein the electrochemical sensor is further configured to perform at least one additional operation from among the following:
depleting a chemical species in the flow of sweat in the microfluidic channel (9), said chemical species having an oxidation potential lower than the oxidation potential of nitric oxide, and
producing a signal that is representative of a flow rate of the flow of sweat in the microfluidic channel (9).

2. The detection apparatus as claimed in claim 1, in which the structure is a multilayer structure (1) comprising a lower layer (3) and at least one layer atop the lower layer (3), the microfluidic circuit (8) extending parallel to the lower layer (3), and the lower layer (3) comprising said entry orifice (4).

3. The detection apparatus as claimed in claim 2, in which the multilayer structure (1) comprises an upper layer (7) and at least one middle layer (6) situated between the lower layer (3) and the upper layer (7), the microfluidic circuit (8) being formed in a thickness of the at least one middle layer (6).

4. The detection apparatus as claimed in claim 3, in which the at least one middle layer comprises a first middle layer (6) and a second, sealing middle layer (26) situated between the first middle layer (6) and the upper layer (7), the second, sealing middle layer (26) comprising an opening (28) at the electrodes.

5. The detection apparatus as claimed in claim 2, in which the multilayer structure (1) comprises an upper layer (7) and an outlet orifice (13) traversing the upper layer (7), in which the at least one microfluidic channel (9) is in communication with the outlet orifice (13).

6. The detection apparatus as claimed in claim 3, in which the at least four electrodes are disposed on an inner face of the upper layer (7) closing the microfluidic channel (9) at a top and/or on an upper face of the lower layer (3) closing the microfluidic channel (9) at a bottom.

7. The detection apparatus as claimed in claim 1, in which, in the direction of the flow (98), the at least four electrodes comprise in succession the first working electrode (20), in the form of a depletion electrode, the second working electrode (23) for measuring the concentration of nitric oxide, and the counter-electrode, the reference electrode (21) being placed at a position immediately upstream of the first working electrode (20) or immediately downstream of the second working electrode (23).

8. The detection apparatus as claimed in claim 1, in which, in the direction of the flow (98), the at least four electrodes comprise in succession the first working electrode (20) for measuring the concentration of nitric oxide, the second working electrode (23) for measuring the concentration of nitric oxide, and the counter-electrode, the reference electrode (21) being placed at a position immediately upstream of the first working electrode (20) or immediately downstream of the second working electrode (23).

9. The detection apparatus as claimed in claim 7, in which the electrochemical sensor (10) is configured to produce the signal that is representative of the flow rate by measuring a delay (Δt) between a variation in current in the first working electrode (20) and a variation in current in the second working electrode (23).

10. The detection apparatus as claimed in claim 1, in which the electrochemical sensor (10) is configured to produce a signal that is representative of instantaneous production of nitric oxide in the investigation zone (97) on the basis of the signal that is representative of the concentration of nitric oxide and of the signal that is representative of the flow rate of the flow of sweat (98).

11. The detection apparatus as claimed in claim 1, in which the electrochemical sensor (10) is configured to produce the signal that is representative of the concentration of nitric oxide by an electrical, especially amperometric, measurement between at least one of said working electrodes (20, 23) and the counter-electrode (30).

12. The detection apparatus as claimed in claim 1, in which the electrochemical sensor (10) is configured to polarize at least one of said working electrodes (20, 23) to an electrical potential for oxidation of nitric oxide.

13. The detection apparatus as claimed in claim 1, in which the electrochemical sensor (10) is configured to produce a signal that is representative of a concentration in the flow of sweat of at least one of the following chemical compounds: nitrite ion, hydrogen peroxide and peroxynitrite, dissolved in sweat.

14. The detection apparatus as claimed in claim 13, in which the electrochemical sensor (10) comprises a third working electrode (25) between the first or second working electrode (20, 23) and the counter-electrode for measuring the chemical compound.

15. The detection apparatus as claimed in claim 1, comprising a colorimetric detection device (18) connected to the channel (9) downstream of the electrochemical sensor (10), the colorimetric detection device (18) comprising a hydrophilic porous body impregnated with a chemical reagent capable of reacting with one of the following chemical compounds: nitrite ion, hydrogen peroxide, peroxynitrite, sulfur dioxide, hydrogen sulfide, nitric oxide, carbon monoxide and hypochlorous acid, dissolved in sweat, so as to provide a colored indicator indicating a quantity of said chemical compound in the flow of sweat (98).

16. The detection apparatus as claimed in claim 15, in which the chemical reagent comprises a Griess reagent capable of reacting with the nitrite ion dissolved in the flow of sweat (98).

17. The detection apparatus as claimed in claim 5, comprising a colorimetric detection device (18) connected to the channel (9) downstream of the electrochemical sensor (10), the colorimetric detection device (18) comprising a hydrophilic porous body impregnated with a chemical reagent capable of reacting with one of the following chemical compounds: nitrite ion, hydrogen peroxide, peroxynitrite, sulfur dioxide, hydrogen sulfide, nitric oxide, carbon monoxide and hypochlorous acid, dissolved in sweat, so as to provide a colored indicator indicating a quantity of said chemical compound in the flow of sweat (98); and
wherein the colorimetric detection device (18) is disposed in the outlet orifice (13).

18. The detection apparatus as claimed in claim 1, in which the electrochemical sensor (10) is configured to polarize at least one said working electrode (20, 23, 25) during a determined time with a periodic recurrence.

19. The detection apparatus as claimed in claim 1, in which the microfluidic circuit (8) comprises a plurality of microfluidic channels (9) each guiding a flow of sweat, which are connected in derivation from one another to the entry orifice (4).

20. The detection apparatus as claimed in claim 19, in which the plurality of microfluidic channels (9) comprises an additional microfluidic channel (9) comprising an electrochemical sensor (10), the electrochemical sensor (10) comprising at least three electrodes disposed successively in a longitudinal direction of the additional microfluidic channel (9), the at least three electrodes comprising a reference electrode (21), a counter-electrode (30) and at least one working electrode (20, 23, 25), the additional electrochemical sensor (10) being configured to polarize the electrodes to an electrical potential for oxidation of a chemical compound selected from nitrite ion, hydrogen peroxide and peroxynitrite and being configured to produce at least one signal that is representative of a concentration of said chemical compound dissolved in a flow of sweat in the additional microfluidic channel (9).

21. The detection apparatus as claimed in claim 19, in which the plurality of microfluidic channels comprises an additional microfluidic channel (109) comprising a colorimetric detection device (18), the colorimetric detection device (18) comprising a hydrophilic porous body impregnated with a chemical reagent capable of reacting with one of the following chemical compounds: nitrite ion, hydrogen peroxide, peroxynitrite, sulfur dioxide, hydrogen sulfide, nitric oxide, carbon monoxide and hypochlorous acid, so as to provide a colored indicator indicating a concentration or a quantity of the chemical compound dissolved in a flow of sweat in the additional microfluidic channel (109).

22. The detection apparatus as claimed in claim 21, in which the additional channel (109) comprises a chrono-sampling system connected to the entry orifice (4), the chrono-sampling system including a plurality of chambers configured to fill sequentially with sweat, and in which a plurality of colorimetric detection devices (18) are disposed in said chambers, each colorimetric detection device (18) comprising a chemical reagent capable of reacting with a chemical compound, such that the colorimetric detection devices disposed in said chambers provide a colored indicator indicating a cumulative quantity of said chemical compound in the flow of sweat in the additional microfluidic channel (109).

23. The detection apparatus as claimed in claim 15, comprising an optical sensor configured to produce a measurement signal that is representative of an intensity of a color of the chemical reagent in the visible or ultraviolet spectrum.

24. The detection apparatus as claimed in claim 1, comprising a communication device (17) configured to transmit one or more measurement signals produced by the detection apparatus (100) to a storage or post-processing apparatus.

25. A portable device comprising a detection apparatus (100) as claimed in claim 1, the portable device being implemented in the form of: a watch, a telephone, a fabric, a headband, a garment or an undergarment.
